(19) United States
(12) Patent Application Publication
(10) Pub. No.: US 2015/0194744 A1
(43) Pub. Date: Jul. 9, 2015

(54) TATTOOING APPARATUS AND CLIP CORD ASSEMBLY FOR ELECTRICALLY CONNECTING A POWER SUPPLY TO A TATTOO MACHINE

(71) Applicant: Edward James Slocum, Tucson, AZ (US)

(72) Inventor: Edward James Slocum, Tucson, AZ (US)

(21) Appl. No.: 14/148,169

(22) Filed: Jan. 6, 2014

(51) Int. Cl. H01R 4/48; H01R 13/717; A61M 37/00; H01R 13/24

(52) U.S. Cl. CPC H01R 4/48; H01R 13/2407; H01R 13/7175; A61M 37/0076

(57) ABSTRACT

A tattooing apparatus includes a tattoo machine, a power supply electrically connected to the tattoo machine via a clip cord assembly, a switch electrically connected to the power supply to engage the power supply to supply electrical power to the tattoo machine via the clip cord assembly, and the clip cord assembly includes a power indicator light that illuminates only when the clip cord assembly is receiving electrical power.

### FIELD OF THE INVENTION

The present invention relates to tattoos and, more particular, to tools used to by a tattoo artist for marking the skin with indelible markings.

### BACKGROUND OF THE INVENTION

A tattoo is a form of body modification formed by injecting indelible ink into the dermis layer of the skin so as to produce indelible patterns, pictures, legends, or other markings. A tattoo artist is an individual who applies tattoos. A tattoo artist must be artistic, have the ability to draw, work well with people, have the physical endurance to sit for long periods, have the mental acuity to concentrate for long periods of time, and be skilled in the essentials of health and hygiene for ensuring customer safety.

According to modern practice, tattoos are applied with a tattoo machine, which is a hand-held device used to create a tattoo, namely, a permanent marking of the skin with indelible ink. Modern tattoo machines incorporate an electromagnetic mechanism that repeatedly pushes ink into the skin via a barred needle grouping. The tattoo machine is connected to a bench power supply with a clip cord that transfers electrical power from the bench power supply to the tattoo machine. The clip cord consists of a power cord connected between a plug for making an electrical connection when inserted into a jack of a power supply, and a clip or clip assembly for making an electrical connection when clipped to the tattoo machine. And so the plug is plugged into the power supply, and the clip is clipped to the tattoo machine, which the tattoo artist holds in his hand to apply tattoos in the normal manner. Typically, a foot switch is used to activate the tattoo machine.

The clip of the clip cord is clipped to the tattoo machine. The clip cord is part of a circuit that supplies power to the tattoo machine from the power supply via the foot switch. Occasionally, instrument malfunctions result in a partial or complete failure in the operation of the tattoo machine. In the event of a partial or complete failure of the operation of the tattoo machine, action must be taken quickly to resume the tattooing operation to avoid the person receiving the tattoo from experiencing discomfort, and to prevent the tattoo artist from losing his or her concentration and becoming frustrated.

When an intermittent or complete failure in the operation of the tattoo machine is experienced, the tattoo artist is forced to troubleshoot the problem by checking the power supply, checking the foot switch, checking the plug of the clip cord to determine whether it is properly plugged into the power supply, checking the tattoo machine to determine whether it is in need of repair or replacement, replacing the tattoo machine with a new or different tattoo machine, etc. This troubleshooting process interrupts the tattooing process and is time consuming and frustrating, thereby necessitating certain new and useful improvements in the art.

### SUMMARY OF THE INVENTION

According to the principle of the invention, a tattooing apparatus includes a tattoo machine, a power supply electrically connected to the tattoo machine via a clip cord assembly, a switch electrically connected to the power supply to engage the power supply to supply electrical power to the tattoo machine via the clip cord assembly, and the clip cord assembly includes a power indicator light that illuminates only when the clip cord assembly is receiving electrical power. The clip cord assembly includes a power cord electrically connecting the power supply to a clip assembly electrically connected to the tattoo machine. The clip assembly includes a yoke having a first end formed with a first prong, a second end formed with a second prong, and a bight connecting the first and second ends, and the first and second prongs are electrically connected to the tattoo machine. The power indicator light is attached to the yoke. More specifically, the power indicator light is attached to the bight of the yoke such that when illuminated directs illumination toward the first and second prongs and the tattoo machine. The power indicator light is a light emitting diode, and the yoke has shape memory.

According to the principle of the invention, a clip cord assembly for electrically connecting a power supply to a tattoo machine includes a power cord electrically connecting a plug to a clip assembly, the plug for electrically connecting a power supply, and the clip assembly for electrically connecting a tattoo machine, and the clip assembly includes a power indicator light electrically connected to the power cord that illuminates only when the clip assembly is receiving electrical power via the power cord. The clip assembly includes a yoke having a first end formed with a first prong, a second end formed with a second prong, and a bight connecting the first and second ends, the first and second prongs for electrically connecting the tattoo machine. The power indicator light is coupled to the yoke. More specifically, the power indicator light is coupled to the bight of the yoke such that when illuminated directs illumination toward the first and second prongs. The power indicator light is a light emitting diode, and the yoke has shape memory.

According to the principle of the invention, a combination clip cord assembly and tattoo machine includes a tattoo machine electrically connected to a clip cord assembly for electrically connecting the tattoo machine to a power supply, and the clip cord assembly includes a power indicator light that illuminates only when the clip cord assembly is receiving electrical power. The clip cord assembly includes a power cord for electrically connecting the power supply to a clip assembly electrically connected to the tattoo machine. The clip assembly includes a yoke having a first end formed with a first prong, a second end formed with a second prong, and a bight connecting the first and second ends, and the first and second prongs electrically connected to the tattoo machine. The power indicator light is attached to the yoke. More specifically, the power indicator light is attached to the bight of the yoke such that when illuminated directs illumination toward the first and second prongs and the tattoo machine. The power indicator light is a light emitting diode, and the yoke has shape memory.

### BRIEF DESCRIPTION OF THE DRAWINGS

Referring to the drawings:

FIG. 1 is a highly generalized schematic representation of a tattooing apparatus including a clip cord assembly and a switch each electrically connected to a power supply, the clip cord assembly is constructed and arranged in accordance with the principle of the invention and is for electrically connecting the power supply to a tattoo machine;

FIG. 2 is a right perspective view of a clip assembly of the clip cord assembly of FIG. 1;

FIG. 3 is a front perspective view of the embodiment of FIG. 1;

FIG. 4 is a top plan view of the embodiment of FIG. 1;

FIG. 5 illustrates the embodiment of FIG. 1 as it would appear electrically connected to a tattoo machine; and

FIG. 6 is a right perspective view of a clip assembly of a clip cord assembly, the clip assembly is for electrically connecting a tattoo machine and is constructed and arranged in accordance with an alternate embodiment of the invention.

### DETAILED DESCRIPTION

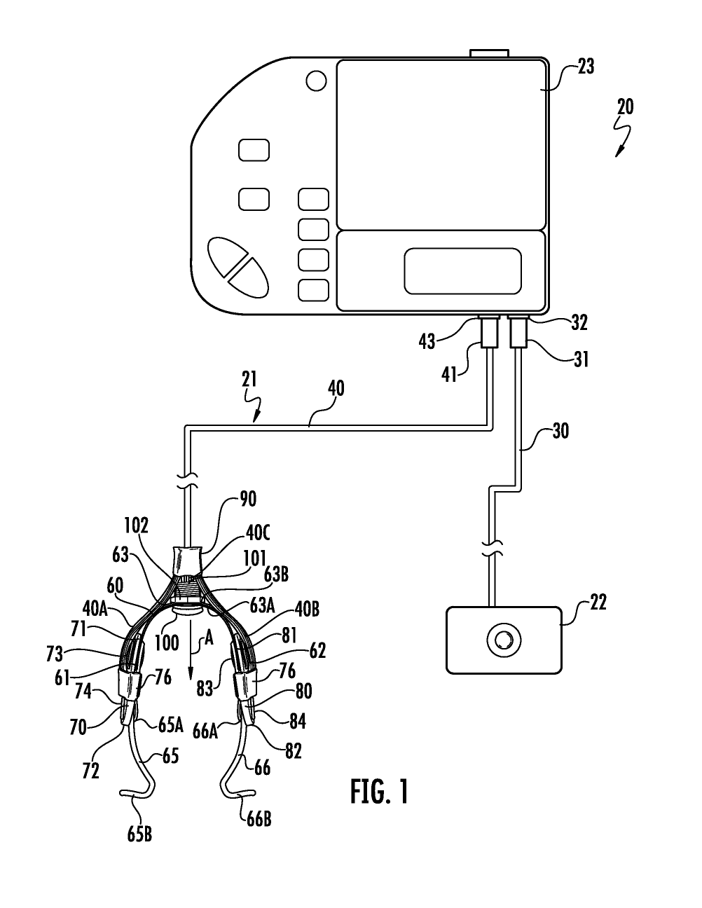

A tattoo is a form of body modification formed by injecting indelible ink into the dermis layer of the skin so as to produce indelible patterns, pictures, legends, or other markings. Turning now to the drawings, in which like reference characters indicate corresponding elements throughout the several views, attention is first directed to FIG. 1 in which there is seen a highly generalized schematic representation of a tattooing apparatus 20 useful by a tattoo artist in the application of tattoos. Tattooing apparatus 20 includes a clip cord assembly 21 and a switch 22 each electrically connected to a power supply 23. Clip cord assembly 21 is constructed and arranged in accordance with the principle of the invention and is for electrically connecting power supply 23 to a tattoo machine.

Switch 22 is a well-known foot switch operated by the foot of a tattoo artist. Switch 22 is electrically connected to power supply via power cord 30. Power cord 30 has a plug 31, which in this case is a conventional mono jack. Power cord 30 electrically connects plug 31 to switch 22. Plug 31 is plugged into a corresponding electrical socket 32 of power supply 23 to electrically connect switch 22 to power supply 23. Switch 22, power supply 23, power cord 30, and plug 31 are entirely conventional and well-known to the skilled tattoo artist, further details of which will not be discussed in further detail.

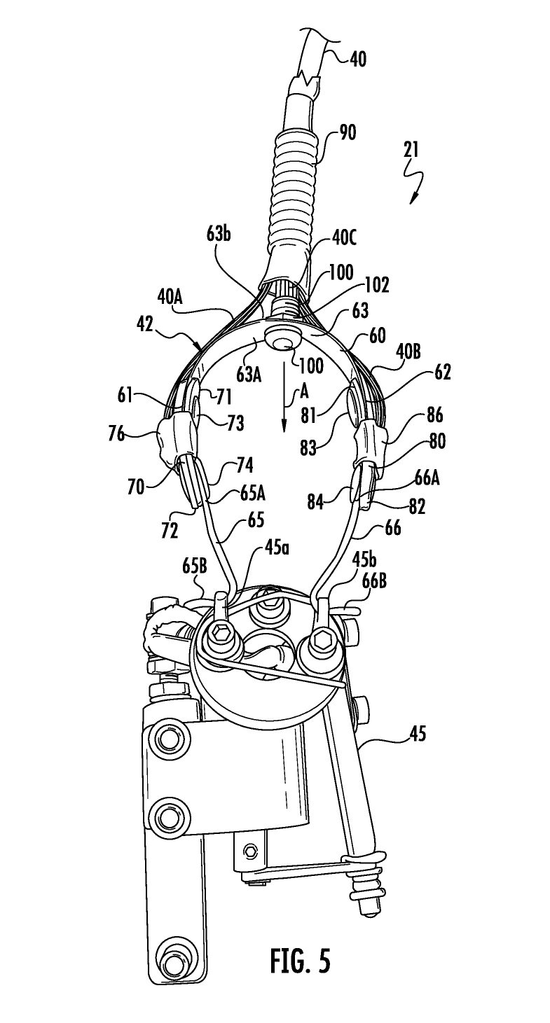

Clip cord assembly 21 includes power cord 40 that electrically connects a plug 41 to a clip assembly 42. Plug 41 is for electrically connecting power supply 23, and clip assembly 42 is for electrically connecting tattoo machine 45 as illustrated in FIG. 5. Tattoo machine 45 is entirely conventional, the details which are well-known in the art and are not described. In this example, plug 41 is a conventional mono jack, and power cord 40 electrically connects plug 41 to clip assembly 42. Plug 41 is plugged into a corresponding electrical socket 43 of power supply 23 to electrically connect clip assembly 42 to power supply 23. Switch 22 electrically connected to power supply via power cord 30 is used in the normal manner to engage power supply 23 to supply electrical power to a tattoo machine via clip cord assembly 21 electrically connecting power supply 23 to the tattoo machine. And so tattooing apparatus 20 includes tattoo machine 45 in FIG. 5, and in FIG. 1 power supply 23 electrically connected to tattoo machine 45 of FIG. 5 via clip cord assembly 21, and switch 22 in FIG. 1 electrically connected to power supply 23 to engage power supply 23 to supply electrical power to tattoo machine 45 of FIG. 5 via the clip cord assembly of FIG. 1. Clip cord assembly 21 is part of a circuit that supplies power to tattoo machine 45 from power supply 23 via switch 22. For reference purposes, this circuit is herein defined as a tattooing circuit.

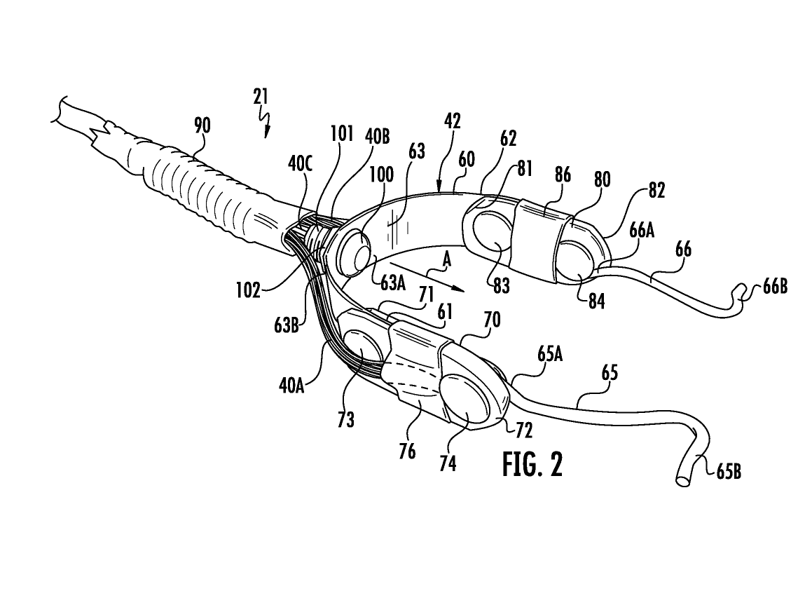

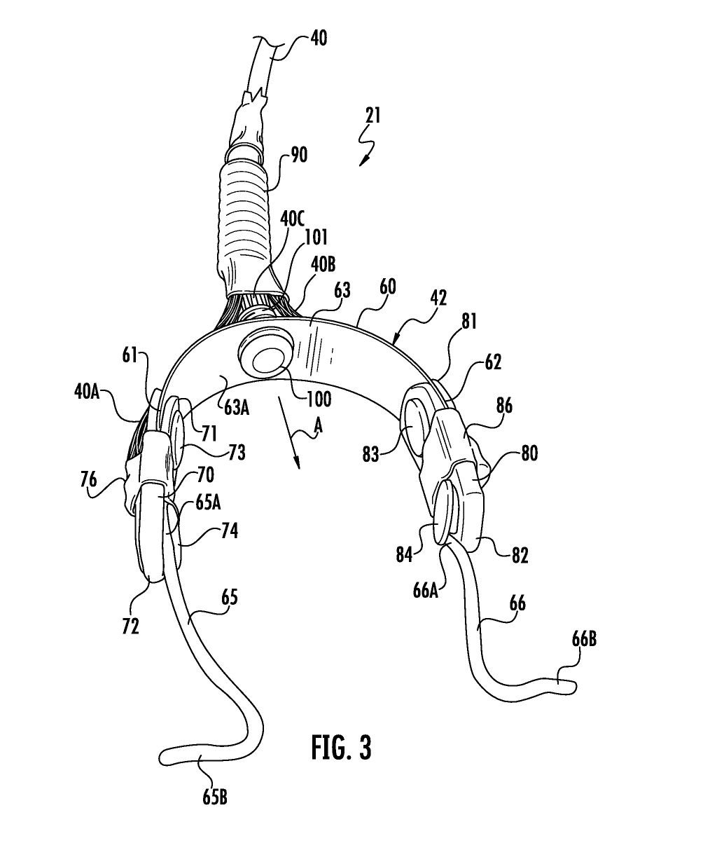

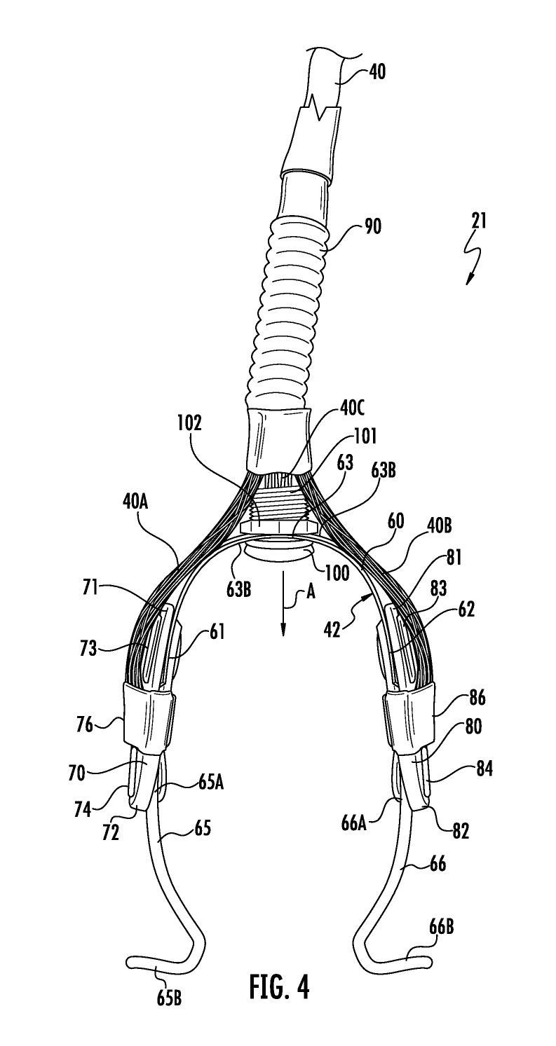

Referring to FIGS. 2, 3, and 4, clip assembly 42 includes yoke 60, which is a U-shaped member having first end 61, second end 62 opposite to first end 61, and bight 63 connecting first and second ends 61 and 62. Yoke 60 has shape memory, and is constructed of a material having a shape memory property, such as spring steel. The shape memory of yoke 60 allows yoke 60 to be flexed under stress, so as to move first and second ends 61 and 62 outwardly and inwardly relative to each other, and to assume its original shape when free of a deforming stress and this characterizes the shape memory property of yoke 60. First end 61 of yoke 60 is formed with a first prong 65, and second end 62 of yoke 60 is formed with a second prong 66. First and second prongs 65 and 66 are the mirror image of one another. First prong 65 is opposite to, and generally parallel with respect to, second prong 66. First and second prongs 65 and 66 are electrically connected to power cord 40, and are for electrically connecting tattoo machine 45 in FIG. 5 via clipping.

First end 61 of yoke 60 is formed with an attached first connector 70, and second end 61 of yoke 60 is formed with an attached second connector 80. First connector 70 connects first prong 65 to first end 61 of yoke 60, and second connector 80 connects second prong 66 to second end 62 of yoke 60. First and second connectors 70 and 80 are substantially parallel with respect to one another.

First connector 70 has proximal end 71 and opposed distal end 72. Proximal end 71 is connected to first end 61, and extends outward to distal end 72. Proximal end 71 is bifurcated. First end 61 of yoke 60 is inserted between the bifurcations of proximal end 71, and is connected to proximal end 71 via rivet 73. First prong 65 is elongate and has inner end 65A and opposed pronged outer end 65B, which extends laterally outward in a direction that is transverse relative the length of first prong 65 extending from inner end 65A to pronged outer end 65B. Inner end 65A is coupled to distal end 72 of first connector 70 with rivet 74. First prong 65 extends outwardly from inner end 65A attached to distal end 72 of first connector 70 to pronged outer end 65A, which is available to be electrically connected to tattoo machine 45 via clipping as in FIG. 5. First connector 70 is formed of an electrically insulative material, such as plastic, ceramic, hard rubber, or the like, and rivets 73 and 74 are each fashioned of an electrically conductive material, such as brass. First prong 65 is fashioned of an electrical conductive material, such as spring steel. Inner end 65A is looped around the shank of rivet 74 and is captured between an inner head of rivet 74 and an inner side of first connector 70. Inner end 65A is in direct contact with rivet 74 and is thus conductively connected to rivet 74.

Identically to first connector 70, second connector 80 has proximal end 81 and opposed distal end 82. Proximal end 81 is connected to second end 62, and extends outward to distal end 82. Proximal end 81 is bifurcated. Second end 62 of yoke 60 is inserted between the bifurcations of proximal end 81, and is connected to proximal end 81 via rivet 83. Second prong 66 is elongate and has inner end 66A and opposed pronged outer end 66B. Inner end 66A is coupled to distal end 82 of second connector 80 with rivet 84. Second prong 66 extends outwardly from inner end 66A attached to distal end 82 of second connector 80 to pronged outer end 66A, which is available to be electrically connected to tattoo machine 45 via clipping as in FIG. 5. Pronged outer end 66A extends laterally outward in a direction that is opposite to that of pronged end 65B of first prong 65 and that is transverse relative the length of second prong 66 extending from inner end 66A to pronged outer end 66B. Second connector 80 is formed of an electrically insulative material, such as plastic, ceramic, hard rubber, or the like, and rivets 83 and 84 are each fashioned of an electrically conductive material, such as brass. Second prong 66 is fashioned of an electrical conductive material, such as spring steel. Inner end 66A is looped around the shank of rivet 84 and is captured between an inner head of rivet 84 and an inner side of second connector 80. Inner end 66A is in direct contact with rivet 84 and is thus conductively connected to rivet 84.

First and second prongs 65 and 66 are electrically connected to power cord 40. At clip assembly 42 power cord 40 is split into a first cord part 40A electrically connected to first prong 65, and a second cord part 40B electrically connected to second prong 66. First cord part 40A is a positive part that supplies a positive polarity, and second cord part 40B is a negative part that supplies a negative polarity. First cord part 40A extends along the outer side of first end 61 of yoke 60 and along the outer side of first connector 70 to rivet 74, where it is looped around the shank of rivet 74 and is captured between an outer head of rivet 74 and an outer side of first connector 70. First cord part 40A is in direct contact with rivet 84 and is thus conductively connected to rivet 84 which, in turn, electrically connects first cord part 40A to first prong 65. Shrink tubing 76 concurrently encircles first connector 70 and first cord part 40A between rivets 73 and 74, which helps to secure first cord part 40A in place along the outer side of first connector 70.

Second cord part 40B extends along the outer side of second end 62 of yoke 60 and along the outer side of second connector 80 to rivet 84, where it is looped around the shank of rivet 84 and is captured between an outer head of rivet 84 and an outer side of second connector 80. Second cord part 40B is in direct contact with rivet 84 and is thus conductively connected to rivet 84 which, in turn, electrically connects second cord part 40B to second prong 66. Shrink tubing 86 concurrently encircles second connector 80 and second cord part 40B between rivets 83 and 84, which helps to secure second cord part 40B in place along the outer side of second connector 80.

The electrical connection between first cord part 40A and first prong 65 and second cord part 40B to second prong 66 characterizes the electrically connection of first and second prongs 65 and 66 to power cord 40. When pronged outer ends 65B and 66B are electrically connected to tattoo machine 45 via clipping as in FIG. 5, the positive side of an electric current is ready to be applied to first prong 65 via first cord part 40A and the negative side of the electric current is ready to be applied to second prong 66 via second cord part 40B so as to power tattoo machine 45 in the normal manner. To electrically connect clip assembly 42 of clip cord assembly 21 to tattoo machine 45 in FIG. 5,

To electrically connect clip cord assembly 21 to tattoo machine 45 in FIG. 5, clip assembly 42 is electrically connected to tattoo machine 45. To do this, yoke 60 is flexed, such as by hand, so as to concurrently move first and second ends 61 and 62 and first and second prongs 65 and 66 inwardly toward one another narrowing the gap between pronged ends 65B and 66B, pronged ends 65A and 66A are aligned with, and threaded through, corresponding eyelet contacts 45A and 45B of tattoo machine 66, and yoke 60 is then released causing yoke 60 to tend to assume its original shape, whereby first and second arms 61 and 62 urge outwardly so as to concurrently urge pronged outer ends 65B and 66B through the respectively eyelet contacts 45A and 45B and against the inner sides of the respective eyelet contacts 45A and 45B. This clips pronged outer ends 65B and 66B to the corresponding eyelet contacts 45A and 45B and concurrently electrically connects pronged outer ends 65B and 66B to the corresponding eyelet contacts 45A and 45B thereby electrically connecting clip assembly 42 to tattoo machine 45 via clipping thereby electrically connecting tattoo machine 45 to clip cord assembly 21. Tattoo machine 45 is decoupled from clip assembly 42 simply by reversing this operation.

In FIGS. 1-5, clip cord assembly 42 includes a power indicator light 100 that illuminates only when clip cord assembly 42 is receiving electrical power from power supply 23 via the activation of switch 22 in FIG. 1. As clip cord assembly 21 is part of the tattooing circuit that supplies power to the tattoo machine from the power supply via switch 22, light 100 when illuminated is indicative that this tattooing circuit is closed, and light 100 when un-illuminated is indicative that this tattooing circuit is open. When the tattooing circuit is closed, clip cord assembly 21 is powered for powering tattoo machine 45 when electrically connected to clip cord assembly 21. When the tattooing circuit is open, clip cord assembly 21 is unpowered rendering unpowered tattoo machine 45 electrically connected to clip cord assembly 21.

Tattoo machine 45 is electrically connected to clip cord assembly 21 via clip assembly 42 in FIG. 5 for electrically connecting tattoo machine 45 to power supply of FIG. 1, and clip cord assembly 21 includes light 100 that illuminates only when clip cord assembly 21, which includes clip assembly 42 of clip cord assembly 21, is receiving electrical power or, in other words, only when the tattooing circuit is closed such that clip cord assembly 21 is powered. Power indicator light 100, which is preferably a light emitting diode (LED), is electrically connected to power cord 40, specifically, to third cord part 40C of power cord 40, which supplies positive and negative polarities to light 100 so as to power light 100 in a normal manner resulting in it being illuminated. In this embodiment, light 100 is attached to yoke 60. More specifically, light 100 is attached to bight 63 of yoke such that when illuminated directs illumination the direction of arrowed line A in FIG. 5 toward first and second prongs 65 and 66, and to tattoo machine 45 electrically connected to first and second prongs 65 and 66 in FIG. 5. In FIGS. 2-5, the portion of power cord 40 leading to light 100 and bight 62 is encircled by a coil spring (not shown) that is, in turn, encircled by shrink tubing 90. This arrangement protects power cord 40 and stabilizes power cord 40 to prevent power cord 40 from bending and shorting prematurely.

Light 100 is positioned along an inner side 63A of bight 63 of yoke 60 in FIGS. 1-5. In FIG. 5, part 40C of power cord 40 extends through an externally threaded ring 101 located along outer side 65B of bight 63, and is electrically connected to light 100 on outer side 63A of bight 63. An internally threaded nut 102 is threaded on externally threaded ring 101, and is tightened via rotation directly against outer side 63B of bight 63 to clamp nut 102 and light 100 against the respective inner and outer sides 63B and 63A of bight 63. This secures light 100 to bight 63 of yoke 60 along outer side 63A of bight 63 facing prongs 65 and 66 in FIGS. 1-5 and tattoo machine 45 connected to prongs 65 and 66 in FIG. 5.

According then to the principle of the invention, power cord 40 electrically connects plug 41 to clip assembly 42, plug 41 is for electrically connecting power supply 23, clip assembly 42 is for electrically connecting tattoo machine 45, and clip assembly 42 includes light 10 electrically connected to part 40C of power cord 40 and that illuminates only when clip cord assembly 21, which includes clip assembly 42, is receiving electrical power via power cord 40 or, in other words, only when the tattooing circuit is closed such that clip cord assembly 21 is powered. Tattooing apparatus 20, in turn, includes tattoo machine 45 in FIG. 5 electrically connected to clip cord assembly 21 via clip assembly 42, and in FIG. 1 power supply 23 electrically connected to tattoo machine 45 of FIG. 5 via clip cord assembly 21, and switch 22 in FIG. 1 electrically connected to power supply 23 to engage power supply 23 to supply electrical power to tattoo machine 45 of FIG. 5 via the clip cord assembly of FIG. 1. Upon engaging power supply 23 via foot switch 22, electrical power transmitted tattoo machine 45 via clip cord assembly 21 concurrently powers tattoo machine 45, for operation in the normal manner in the application of a tattoo, and light 100 causing light 100 to be illuminated. Light 100 when illuminated provides a visual indication that electrical power is being supplied to clip cord assembly 21, including clip assembly 42, and, in other words, provides a visual indication that the tattooing circuit is closed such that clip cord assembly 21 is powered. While clip cord assembly 21, including clip assembly 42, is powered with electrical power, in that tattooing circuit is closed such that clip cord assembly 21 is powered, light 100 remains illuminated. When light 100 is illuminated, the resulting light is directed toward first and second prongs 65 and 66 and tattoo machine 45 electrically connected to first and second prongs 65 and 66, which has the resulting benefit of illuminating the area being tattooed by the tattoo artist using tattoo machine 45.

Upon deactivating switch 22, electrical power from power supply 23 from power supply 23 to clip cord assembly 21 is cut off which, in turn, concurrently deactivates tattoo machine 45 and light 100. While clip cord assembly 21, including clip assembly 42, remain unpowered, in which the tattooing circuit is open such that clip cord assembly 21 is unpowered, light 100 remains un-illuminated. Light 100 being off and un-illuminated provides a visual indication that clip cord assembly 21, including clip assembly 42, is not powered with electrical power from power supply 23, and that the tattooing circuit is open rendering clip cord assembly 21 unpowered.

During operation of tattooing apparatus 20, equipment failures may cause the complete or intermittent failure of the operation of tattoo machine 45. When an intermittent or complete failure in the operation of the tattoo machine is experienced, the tattoo artist is normally forced to troubleshoot the problem by checking power supply 23, checking switch 23, checking clip cord assembly 21 including checking plug 40 to determine whether it is properly plugged into power supply, checking power cord for breaks, checking tattoo machine 45 to determine whether it is in need of repair or replacement, replacing the tattoo machine with a new or different tattoo machine, etc. Again, this troubleshooting process interrupts the tattooing process and is time consuming and frustrating. To alleviate at least some of this frustration, upon an intermittent or complete failure in the operation of tattoo machine 45 during tattooing, an immediate visual inspection of light 100 may be made. If light 100 is illuminated, the tattoo artist may immediately determine that clip cord assembly 21, which includes clip assembly 42, is powered by power supply 23, and that the tattooing circuit is closed such that clip cord assembly 21 is powered. In this situation, the tattoo artist can immediately determine that the intermittent or complete failure in the operation of tattoo machine 45 is due to a partial or complete failure of tattoo machine, which allows the tattoo artist to take immediate steps to repair or replace the subject tattoo machine without the need to troubleshoot the operational fitness of the other components of tattooing apparatus 100. If light 100 is not illuminated or flickers ON and OFF, the tattoo artist may immediately determine that clip cord assembly 21, which includes clip assembly 42, is not receiving power from power supply 23 or is receiving intermittent power from power supply 23, and that the tattooing circuit is closed such that clip cord assembly 21 is powered, or that the tattooing circuit is intermittently closing such that clip cord assembly 21 is intermittently powered in the case of light 100 flickering. In this situation, the tattoo artist can immediately determine that the intermittent or complete failure in the operation of tattoo machine 45 is due to a partial or complete failure of the tattooing circuit or of one or more components of tattooing apparatus 20 other than tattoo machine 45, which prevents the tattoo artist from unnecessarily investigating the operational fitness of tattoo machine 45. This described reduction in troubleshooting allows the tattoo artist to more quickly investigate and solve the intermittent or complete failure of tattoo machine 45 to allow the tattoo artist to quickly and efficiently resume tattooing.

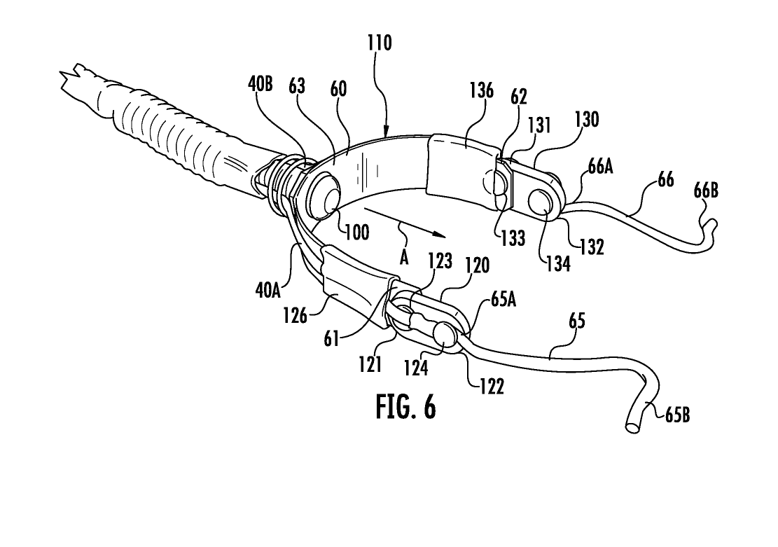

An alternate embodiment of a clip assembly 110 of a clip cord assembly is illustrated in FIG. 6. Like clip assembly 42, clip assembly 110 is for electrically connecting a tattoo machine, such as tattoo machine 45 in FIG. 5. In common with clip assembly 42, clip assembly 110 shares yoke 60 including first end 61 formed with first prong 65 having inner end 65A and pronged outer end 65B, second end 62 formed with second prong 66 having inner end 66A and pronged outer end 66B, bight 63 connecting first and second ends 61 and 62, and light 100 electrically connected to third cord part 40B of power cord 40B. Other than the specific structural features of clip assembly 110 discussed briefly below, the operation of clip assembly 110 with in a clip cord constructed and arranged in accordance with the principle of the invention is identical to clip assembly 42 in every respect.

First end 61 of yoke 60 is formed with an attached first connector 120, and second end 61 of yoke 60 is formed with an attached second connector 130. First connector 120 connects first prong 65 to first end 61 of yoke 60, and second connector 130 connects second prong 66 to second end 62 of yoke 60.

First connector 120 has proximal end 121 and opposed distal end 122. Proximal end 121 is connected to first end 61 of yoke 60, and extends outward to distal end 122. Proximal end 121 is applied to the outer side of first end 61, and proximal end 121 is connected to first end 61 with rivet 123. First prong 65 is elongate and has inner end 65A and opposed pronged outer end 65B, which extends laterally outward in a direction that is transverse relative the length of first prong 65 extending from inner end 65A to pronged outer end 65B. Inner end 65A is coupled to distal end 122 of first connector 120 with rivet 124. First prong 65 extends outwardly from inner end 65A attached to distal end 122 of first connector 120 to pronged outer end 65A, which is available to be electrically connected to tattoo machine 45 via clipping as in FIG. 5. First connector 120 is formed of an electrically insulative material, such as plastic, ceramic, hard rubber, or the like, and rivets 123 and 124 are each fashioned of an electrically conductive material, such as brass. As in clip assembly 42, first prong 65 is fashioned of an electrical conductive material, such as spring steel. Inner end 65A is looped around the shank of rivet 124 and is captured between an outer head of rivet 124 and an outer side of first connector 120. Inner end 65A is in direct contact with rivet 124 and is thus conductively connected to rivet 124.

Identically to first connector 120, second connector 130 has proximal end 131 and opposed distal end 132. Proximal end 131 is connected to second end 61 of yoke 60, and extends outward to distal end 132. Proximal end 131 is applied to the outer side of second end 61, and proximal end 131 is connected to second end 62 with rivet 133. Second prong 66 is elongate and has inner end 66A and opposed pronged outer end 66B. Inner end 66A is coupled to distal end 132 of second connector 130 with rivet 134. Second prong 66 extends outwardly from inner end 66A attached to distal end 132 of second connector 130 to pronged outer end 66A, which is available to be electrically connected to tattoo machine 45 via clipping as in FIG. 5. Second connector 130 is formed of an electrically insulative material, such as plastic, ceramic, hard rubber, or the like, and rivets 133 and 134 are each fashioned of an electrically conductive material, such as brass. As in clip assembly 42, second prong 66 is fashioned of an electrical conductive material, such as spring steel. Inner end 66A is looped around the shank of rivet 134 and is captured between an outer head of rivet 134 and an outer side of second connector 130. Inner end 66A is in direct contact with rivet 134 and is thus conductively connected to rivet 134.

As in clip assembly 42, first and second prongs 65 and 66 are electrically connected to power cord 40. At clip assembly 42 power cord 40 is split into first cord part 40A, which is sheathed in FIG. 6, electrically connected to first prong 65, and the second cord part 40B, which is also sheathed in FIG. 6, electrically connected to second prong 66. First cord part 40A is the positive lead, and second cord part 40B is the negative lead. First cord part 40A extends along the outer side of first end 61 of yoke 60 and therebeyond along the outer side of first connector 120 over rivet 123 to rivet 124, where it is looped around the shank of rivet 124 and is captured between the outer head of rivet 124 and the outer side of first connector 120. First cord part 40A is in direct contact with rivet 124 and is thus conductively connected to rivet 124 which, in turn, electrically connects first cord part 40A to first prong 65. Shrink tubing 126 encircles first end 61 and first cord part 40A, which helps to secure first cord part 40A in place along the outer side of first connector 120.

Second cord part 40B extends along the outer side of second end 62 of yoke 60 and therebeyond along the outer side of second connector 130 over rivet 133 to rivet 134, where it is looped around the shank of rivet 134 and is captured between the outer head of rivet 134 and the outer side of first connector 120. Second cord part 40B is in direct contact with rivet 134 and is thus conductively connected to rivet 134 which, in turn, electrically connects second cord part 40B to second prong 66. Shrink tubing 126 encircles second end 62 and second cord part 40B, which helps to secure second cord part 40B in place along the outer side of second connector 130.

The invention has been described above with reference to preferred embodiments. However, those skilled in the art will recognize that changes and modifications may be made to the embodiments without departing from the nature and scope of the invention. Various changes and modifications to the embodiments herein chosen for purposes of illustration will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. A tattooing apparatus, comprising:
a tattoo machine;
a power supply electrically connected to the tattoo machine via a clip cord assembly;
a switch electrically connected to the power supply to engage the power supply to supply electrical power to the tattoo machine via the clip cord assembly; and
the clip cord assembly includes a power indicator light that illuminates only when the clip cord assembly is receiving electrical power.

2. The tattooing apparatus according to claim 1, wherein the clip cord assembly includes a power cord electrically connecting the power supply to a clip assembly electrically connected to the tattoo machine.

3. The tattooing apparatus according to claim 1, wherein the clip assembly includes a yoke having a first end formed with a first prong, a second end formed with a second prong, and a bight connecting the first and second ends, and the first and second prongs electrically connected to the tattoo machine.

4. The tattooing apparatus according to claim 3, wherein the power indicator light is attached to the yoke.

5. The tattooing apparatus according to claim 4, wherein the power indicator light is attached to the bight of the yoke such that when illuminated directs illumination toward the first and second prongs and the tattoo machine.

6. The tattooing machine according to claim 5, wherein the power indicator light is a light emitting diode.

7. The tattooing machine according to claim 3, wherein the yoke has shape memory.

8. A clip cord assembly for electrically connecting a power supply to a tattoo machine, the clip cord assembly comprising:
a power cord electrically connecting a plug to a clip assembly, the plug for electrically connecting a power supply, and the clip assembly for electrically connecting a tattoo machine; and
the clip assembly includes a power indicator light electrically connected to the power cord that illuminates only when the clip assembly is receiving electrical power via the power cord.

9. The clip cord assembly according to claim 8, wherein the clip assembly includes a yoke having a first end formed with a first prong, a second end formed with a second prong, and a bight connecting the first and second ends, the first and second prongs for electrically connecting the tattoo machine.

10. The clip cord assembly according to claim 9, wherein the power indicator light is coupled to the yoke.

11. The clip cord assembly according to claim 10, wherein the power indicator light is coupled to the bight of the yoke such that when illuminated directs illumination toward the first and second prongs.

12. The clip cord assembly according to claim 11, wherein the power indicator light is a light emitting diode.

13. The clip cord assembly according to claim 12, wherein the yoke has shape memory.

14. A combination clip cord assembly and tattoo machine, comprising:
a tattoo machine electrically connected to a clip cord assembly for electrically connecting the tattoo machine to a power supply; and
the clip cord assembly includes a power indicator light that illuminates only when the clip cord assembly is receiving electrical power.

15. The combination according to claim 14, wherein the clip cord assembly includes a power cord for electrically connecting the power supply to a clip assembly electrically connected to the tattoo machine.

16. The combination according to claim 15, wherein the clip assembly includes a yoke having a first end formed with a first prong, a second end formed with a second prong, and a bight connecting the first and second ends, and the first and second prongs electrically connected to the tattoo machine.

17. The combination according to claim 16, wherein the power indicator light is attached to the yoke.

18. The combination according to claim 17, wherein the power indicator light is attached to the bight of the yoke such that when illuminated directs illumination toward the first and second prongs and the tattoo machine.

19. The combination according to claim 18, wherein the power indicator light is a light emitting diode.

20. The combination according to claim 16, wherein the yoke has shape memory.
